(19) United States
(12) Patent Application Publication
(10) Pub. No.: US 2006/0073560 A1
(43) Pub. Date: Apr. 6, 2006

(54) Selective expression in filamentous fungi

(76) Inventors: Kerry Burton, Warwickshire (GB); Michael Challen, Warwickshire (GB); Timothy Elliott, West Sussex (GB); Surapareddy Sreenivasaprasad, Warwickshire (GB); Daniel Eastwood, Coventry (GB); Shannon Molloy, Waterford (IE)

Correspondence Address: GREENLEE WINNER AND SULLIVAN P C, 4875 PEARL EAST CIRCLE, SUITE 200, BOULDER, CO, 80301, US

(21) Appl. No.: 10/533,361

(22) PCT Filed: Oct. 31, 2003
(86) PCT No.: PCT/GB03/04716

§ 371 Date: Oct. 21, 2005

(30) Foreign Application Priority Data
Oct. 31, 2002 (GB) 0225390.4

(51) Int. Cl. C12P 21/06; C12N 15/74; C12N 1/16

(52) U.S. Cl. 435/69.1; 435/254.3; 435/484

(57) ABSTRACT

Gene promoters activated only during development of the fruiting body of filamentous fungi permit expression of large quantities of heterologous genes, even where such are otherwise toxic to the organism.

The present invention relates to the transformation of filamentous fungi with heterologous DNA, methods for such transformation and processes for the collection of the resulting expression products.

Filamentous fungi have an unusual life cycle, in that the majority of the cycle is spent as mycelium, which is a largely hidden network of filaments permeating throughout and over a suitable food source. In the case of Agaricus bisporus, or button mushroom, which is a horticultural adaptation of a wild mushroom, the food source is decomposed leaf litter, in the wild, or generally composted cereal straw, when cultivated, and little signs of growth can be observed above ground level, other than the characteristic smell, until the fruiting bodies, or sporophores, start developing.

Compared with the culture of other vegetables, the commercial production of button mushrooms is complex. They are best grown indoors in controlled environmental conditions in trays, shelves or bags filled with a speciality compost, specifically made by, and for the, the mushroom industry from ingredients including: plant remains, such as cereal straw, corn husks, hay, rape straw; animal manure, such as from horse, chicken, cow, pig; gypsum, and other additives.

Mushroom compost is made by the mixing of the raw ingredients, for example straw, manure and gypsum base, with water, which leads to microbial degradation and heat production. The composting process has two phases. In Phase 1 composting, the mixture is either stacked in a windrow outdoors, or in a barn, and turned every 2 to 3 days over a three-week period, or it is placed in a walled bunker and aerated from below over an 8-10 day period. This compost is then subjected to Phase II composting in an aerated bunker where the temperature is raised to 58-60° C. for approximately 12 hours and held at 50-54° C. for 5 days. The compost is then ready for spawning.

Spawn is, typically, sterilised cereal grain, such as rye or millet, which has been colonised with the mushroom mycelium. Spawn is mixed with the compost and maintained at 24° C. After approximately 17 days at this temperature, the compost is fully colonised by the mycelium.

A casing layer is then applied to the surface of the colonised compost to a depth of approximately 5 cm. Casing normally consists of peat (or peat substitute for example, coir, bark, mineral washing), chalk/lime/sugarbeetlime, water and a small amount of specialised spawn, which may be the mycelium cultured on compost, grain, vermiculite or other substrate. When the surface of the casing layer is colonised with mycelium, the temperature is lowered to 16-18° C., which induces production of mushroom sporophores, or fruiting bodies. From application of the casing to harvesting the first sporophore takes approximately 17 days.

The production of mushrooms occurs periodically, over cycles known as flushes, at 7-10 day intervals. High productivity farms normally crop mushrooms over 3 flushes. The time from the first harvest of the first flush to the last harvest of the third flush is about 21 days.

The time each growing house is occupied with each crop is from after casing and during crop production, or 17+21=38 days, or thereabouts. This allows a growing house to be used for 9 crops per year. High productivity farms routinely have yields of 25 kg/m². Because mushroom production is not light dependent, the crops are grown in layers, either four or six to maximise productivity. Accordingly, the four-layer system is expected to produce 3,200 tonnes per annum in a hectare of growing rooms.

The rapid development of the fruiting body is unique in the vegetable kingdom, and represents a significant target for the expression of suitable genes. If this rapid, volumetric expansion of the fruiting body could be harnessed together with expression of a suitable gene, then this might make possible the production, on an industrial scale, of biological products that currently defy large scale production.

Attempts to express heterologous proteins in filamentous fungi have met with numerous problems, not least being the difficulty associated with transforming the fungi in the first place. For example, WO95/02691 relates to transforming a mushroom mycelium and fruit bodies through methods such as electroporation with suitable vectors. Although this method works on a small scale, it is not especially efficient.

WO98/45455 relates to the possibility of transforming moulds, such as Agaricus bisporus, with the Agrobacterium tumefaciens bacterium, which causes crown gall tumours at the wound site of infected dicotyledonous plants. This bacterium is well known for its ability to transform plants, but it has only recently been established that it can also transform filamentous fungi.

WO 96/41882 discloses the expression of hydrophobins by the hyp A, hyp B, hyp C and hyp D genes. These are naturally occurring fungal products, and are expressed during fruiting. Heterologous expression is suggested, in connection with enhancing the flavour and/or nutritional content of the fruiting bodies.

Mol. Gen. Genet. (1993), 238: 91-96, provides a reporter-gene system in S. commune, for the expression of hydrophobins.

The problem still remains that the expression of heterologous genes in substantial amounts will generally substantially reduce the growth potential of the mycelium and, therefore, the harvest of the fruiting body, and prohibits the expression of any substance which is, in any way, toxic to the growth of the fungus.

Surprisingly, it has now been found that at least three genes are switched on, or otherwise subjected to elevated levels of expression, at around the veil-break stage of fruiting body development, and that heterologous DNA under the control of the expression mechanisms of these fungal genes can be selectively expressed at this stage of development of the fungus, rather than during growth of the mycelium.

Thus, in a first aspect, the present invention provides a filamentous fungus transformed with a heterologous sequence of DNA, the fungus being capable of expressing the heterologous DNA, characterised in that the heterologous DNA is under the control of a filamentous fungus transcription promoter active substantially only during stage 1, or later, of the development of the fruiting body of the fungus.

It is a particular advantage of the present invention that little or no metabolic energy need be diverted from mycelium growth, thereby maximising fruiting body mass and concomitant tissue capable of expressing the heterologous gene once it is switched on.

It is a further advantage of the present invention that little or none of the heterologous gene product is expressed during vegetative growth of the mycelium, thus enabling the production of substantially any substance capable of expression in the filamentous fungus in question, even if that substance, either alone or in combination, results in the death or stasis of the fungus. The promoters allow the synchronous switching on of the gene at a time of rapid growth and high metabolism so that, by the time any potentially toxic effects become apparent, harvestable quantities of the substance are available. Where the gene product is a regulator of mushroom growth, for example, then such considerations are not generally necessary.

It is particularly preferred to harvest the expression product of the heterologous DNA, and the product may be purified, if and as desired, by any suitable means, such as are well known in the art.

Accordingly, the present invention further provides a method for the production of a substance expressible by a DNA sequence, wherein the sequence is operably associated with a filamentous fungus transcription promoter active substantially only during stage 1, or later, of the development of the fruiting body of the fungus, the sequence and promoter being expressibly incorporated in a filamentous fungus, the fungus being cultured to fruition and the product being harvested.

At present, no less than three genes have been identified that are expressed substantially only during development of the fruiting body, and particularly during stages 4 to 7 (veil-break onwards). Without being constrained by theory, it is likely that these genes are associated with the massive water uptake required for the expansion of the fruiting body and its maturation.

The three genes, so far identified, are abst1, rafe and mag2. The expression product of abst1 appears to be involved in the transport of sugars, whilst the expression product of rafe is a putative riboflavin aldehyde forming enzyme. The expression product of mag2 is a so far unidentified morphogenesis associated protein.

The sequences of the three genes, and associated promoter and terminator regions, are given in the accompanying Sequence Listing.

### BRIEF DESCRIPTION OF THE FIGURES

FIG. 1 illustrates the stages of development of the fruiting body of a mushroom fungus, from vegetative mycelium (VM) to Stage 1 through to Stage 7.

FIG. 2A illustrates the level of expression of abst1 during the various stages of development of the fruiting body, from vegetative mycelium (VM) to Stage 1 through to Stage 7. FIG. 2B illustrates the gene expression associated with the various parts of the fruiting body at stage 4. US represents upper stipe; LS represents lower stipe; C represents cap; G represents gill; and S represents skin.

FIG. 3 corresponds to FIG. 2, except showing the expression of rafe. The stages in FIG. 3A are as in FIGS. 1 and 2A, above. In FIG. 3B, the parts of the fruiting body in which expression of rafe are shown, is as in FIG. 2B, above.

FIG. 4 corresponds to FIGS. 2 and 3 above, showing the expression of mag2. The stages and fruiting body parts showing expression are as above.

FIG. 5 shows a construct with restriction enzyme sites represented by numbers 1-6.

FIG. 6 shows a promoter-intron cassette, comprising A. bisporus GPD promoter 5′UTR sequence, an ATG start codon and three naturally occurring introns (in1, in2 and in3). The restriction sites KpnI and NarI are shown. The nucleotide sequence of this cassette is given in SEQ ID NO. 12. The ATG (start codon) and VKV . . . ( )F( ) . . . GRIG(R) have not been included in the complete amino acid sequence.

FIG. 7 shows a leader (L) sequence, the SPR leader sequence, comprising 57 nucleotides (SEQ ID NO. 13) encoding 19 amino acids (SEQ ID NO. 14). This leader sequence is derived from Y13805 deposited as Accession Number Y13805 in the EMBL/GenBank/DDBJ databases (Kingsnorth, C. S., Eastwood, D. C. & Burton, K. S. (2000). Agaricus bisporus partial mRNA for serine proteinase).

FIG. 8 shows a Terminator cassette, corresponding to Figure SEQ ID NO. 35, comprising a A. nidulans trpC terminator 3′UTR and a number of engineered restriction sites (Bam H1, BglII and KpnI).

FIG. 9 shows a construct in a pBluescript plasmid, combining A. bisporus GPD promoter with introns (as detailed above, referred to as P) fused with eGFP gene and A. nidulans trpC terminator (as described above, referred to as T). Engineered KpnI sites are used to excise expression cassette from pBluescript and transfer to Agrobacterium binary vector harbouring hygromycin (hph) resistance selectable marker. T-DNA introduced into A. bisporus by Agro-transfection contains hph and heterologous protein (GFP) as a pair of divergently transcribed genes.

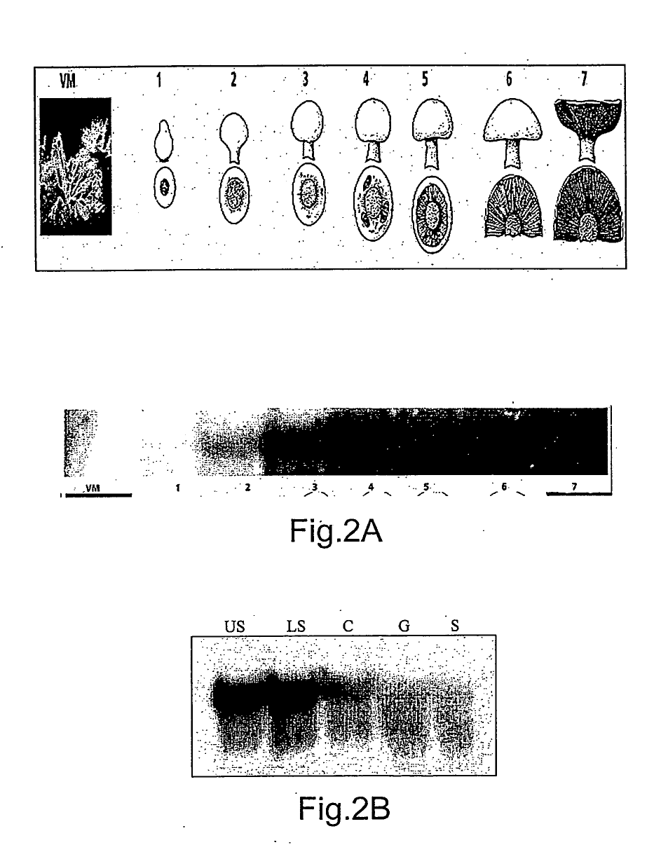

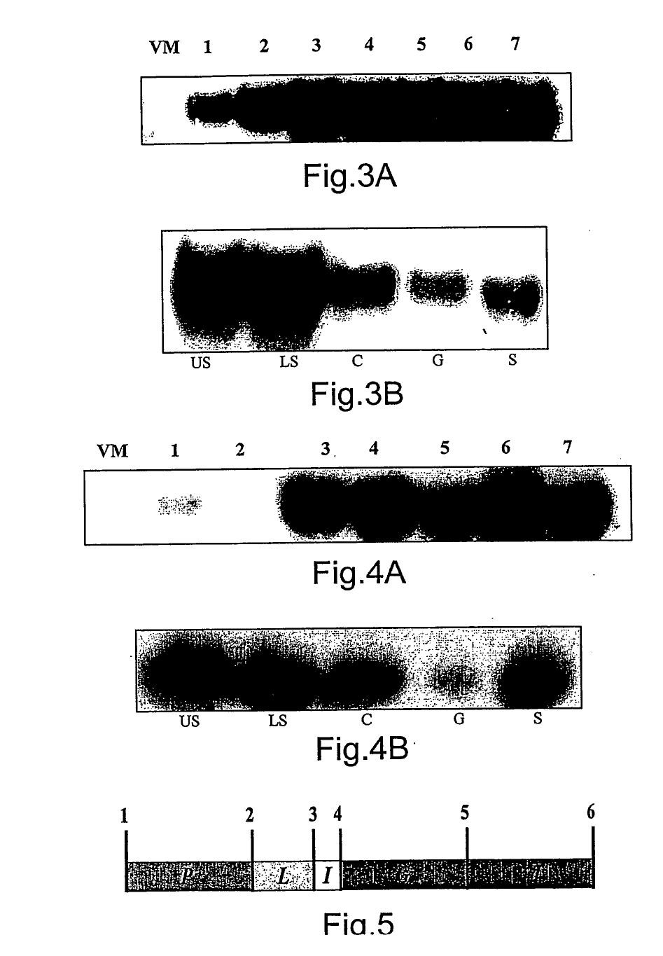

It will be appreciated that the Basidiomycetes, including members of the Agaricales, of which A. bisporus is one, share the exceedingly rapid development of the fruiting body in common. Without being constrained by theory, it is envisaged that this development is as a result of a rapid increase in osmotic pressure in the cells of the immature fruiting body, thereby causing a rapid influx of water into the cells. The resulting sudden expansion of the cells expands the fruiting body up to several hundred times its original size.

One or more sugar transport mechanisms are switched on at the early stages of fruiting body development, and abundant expression product is noted, especially by stage 4. These genes form a preferred subject of the present invention, and especially the control element associated therewith, but it will be appreciated that any gene selectively expressed, or with greatly enhanced expression, during development of the fruiting body is useful in the present invention.

The abst1 gene is up-regulated, by more than 100-fold, during mushroom development, and is abundantly expressed through stages 4 to 7, and represents about 0.6% of the transcripts detected at stage 4. The transcript is about 1 .7 kb in length.

The gene product of rafe is up-regulated by about 50-fold during mushroom development, and is abundantly expressed from stage 4 onwards, as with abst1. The transcript is 0.7 kb in length.

The expression product of mag2 is up-regulated by about 30-fold during mushroom development, and represents about 0.2% of the transcripts at stage 4. Unlike abst1 and rafe, the expression of mag2 appears to be comparable in both the stipe and cap tissues. The length of the transcript is about 0.7kb.

The control elements, and especially the promoters, of these and other genes expressed during the development of the fruiting body are particularly useful in the present invention. Elements from genes associated with sugar transport are particularly preferred.

It is a particular advantage of the present invention that heterologous genes can be expressed at selected stages of sporophore development, where these genes might otherwise be harmful to the fungus. Expression of the heterologous genes occurs substantially only during growth of the fruiting body so that, unless the gene product is acutely toxic, then large amounts of the gene product can be expressed which would otherwise harm or hinder growth of the fungus.

Owing to the commonality of the fruiting body growth process, the promoters of any one filamentous fungus, switched on during the fruiting body growth cycle, may be employed in other filamentous fungi in the context of the present invention, in order to express heterologous genes.

In the accompanying Sequence Listing, SEQ ID NO's 12 and 13 are the promoter sequences associated with abst 1 and rafe, respectively, and the full sequences are provided for abst1 and rafe as SEQ ID NO's 4 and 9. These include a substantial portion of the upstream promoter area and, following the procedures described in the accompanying Example, the upstream promoter area is also obtainable for mag2.

The promoter may be used in association with other suitable control sequences, such as terminators. A suitable terminator may be as shown in the accompanying sequences, or may be the Aspergillus nidulans trpC terminator, for example. Other terminators are well known in the art.

The terminator sequences of abst 1 and rafe are provided as SEQ ID NO's 35 and 36, and it has been found that it is desirable to use terminators generally associated with the promoters, so that it is preferred to use the abst 1 terminator sequence with the abst 1 promoter sequence, and likewise for rafe. However, it will be appreciated that the present invention also envisages any suitable expression system comprising the promoter, and any suitable terminator may be employed, as desired.

It will also be appreciated that the promoter sequences and terminator sequences of the invention are preferably those as listed as SEQ ID NO's 12 and 13, and 35 and 36, respectively, and sequences comprising these sequences, as well as sequences hybridising with these sequences, preferably under conditions of 60% stringency or higher, provided that promoter activity is retained in the by the sequence or the sequence to which it hybridises. Mutations and naturally occurring variants of the sequences are encompassed, and it may be, for example, appropriate to introduce a restriction site or sites for ease of manipulation. Provided that promoter activity is retained, there is no restriction on how much the promoter may be modified. Similar considerations apply to the terminator sequences.

The promoter regions may be used in their entirety when preparing heterologous genes for expression in filamentous fungi. Alternatively, it may be preferred to use consensus sequences from these regions. There is no especial advantage to using consensus sequences, except that these may be shorter. Otherwise, it is sufficient to supply the promoter upstream of the desired heterologous gene. Being a promoter, there is also no requirement that it be in the correct reading frame, just within the appropriate promoter distance.

It is generally preferable that the transformed fungus also expresses a linked selectable marker. Any marker known in the art may be used, and may be excised once a faithful strain has been generated. However, it is generally preferable that the transformed fungus maintains a marker to ensure that the desired heterologous product is still produced, and to ensure that there is no reversion to wild type. In this respect, it is preferred that the marker have no significant negative effect on either the fungus or the product. Such markers may normally be selected from resistance markers, in order that the growth medium contain amounts of an antifungal agent ensuring that only transformed fungus can grow successfully.

Suitable markers include the hygromycin resistance cassette and the benomyl resistance tubulin gene.

Suitable methods for transforming filamentous fungi are as described above with respect to WO95/02691 and WO98/45455, which disclosures are incorporated herein by reference.

In general, the desired control sequences are ligated with the appropriate heterologous expression sequences and prepared for insertion into a suitable preparation of the fungus, such as protoplasts, all by methods well known in the art.

The resulting organism can then be grown by standard methods, and prepared as spawn after cultivation of the resulting mycelium. Spawn has the advantage that it can be stored inert for relatively long periods of up to about a year, although it is generally preferred to use it within about 4 months.

Spawn may be produced in any recognised manner, such as by growing the mycelium on sterile agar and introducing the culture to autoclaved grain. The grains may then be stored at elevated temperature to encourage colonisation, and then kept at reduced temperatures until needed.

It will be appreciated that the heterologous gene for incorporation may be in the form of cDNA or genomic DNA. cDNA is preferred, as it is generally shorter and more easy to handle.

It will also be appreciated that the heterologous gene insert should encode the sequence desired, including leader sequences and cleavage sequences, if required.

It will also be appreciated that greater expression may be achieved if fungal codons are used in place of mammalian codons, although expression will still occur, and such substitution is not necessary.

It will further be appreciated that heterologous genes may need to be expressed in the form of a cassette, for example, in order to produce the required product. In general, it is preferred to require as few heterologous gene products as possible, as the greater the number, the more likely it is that the fungal metabolism will interfere in some way, and it is generally desirable to minimise unpredictability.

Thus, although not limited thereto, it is generally preferred to limit the number of heterologous expression products to one, two or three, preferably one or two, and preferably one, other than any marker. The marker is preferably linked to the heterologous gene, such as downstream of the gene and also under the control of the fungal promoter, so as best to indicate successful and/or continuing stable transformation.

It is particularly preferred that products such as peptides be the target, as these can be harvested relatively simply. Thus, enzymes and antibodies are particularly useful, although conformational proteins, such as vaccine antigens, and active peptides such as interferons are also useful.

Accordingly, heterologous genes suitable for expression in the filamentous fungi include those whose expression results in the production of: antibodies, including other diagnostic material; secondary metabolites, such as lectins, pesticidal compounds such as Bacillus thuringiensis toxin (Bt toxin); therapeutic compounds such as vaccines, steroids, heterocyclic organic compounds; biological macromolecules, such as interferon, endostatin and insulin; and medical enzymes, such as thrombolytics and cerebrosidases.

In the context of the present invention, the term “heterologous” includes native DNA not normally associated with heightened expression during sporophore production. In such a respect, the native gene becomes heterologous insofar as its expression pattern is altered. Such expression may generally serve one of two purposes. The first is generally to obtain large/greater amounts of native protein, such as by transforming the filamentous fungi with extra copies or modified copies of a native gene or genes. The second may be used instead to affect/control the characteristics of mushroom crop production, such as by altering the timing of crop, flushing pattern, yield, growth rate and/or final size of the mushroom sporophore. This latter may also suitably be achieved by the introduction of heterologous DNA from other species, if desired.

The crops are preferably allowed to go to full cap development, where possible, in order to maximise expression of the heterologous gene, although the skilled person will appreciate the best stage for harvesting any given product. The resulting caps may then be processed in any suitable manner to extract and/or purify the product, or the caps may otherwise be employed or processed, as desired.

Where the product is potentially dangerous, standard procedures may be employed between crops to entirely sterilise the area, such as steam sterilisation and swabbing of the walls, as described above.

The present invention will now be illustrated further, by reference to the following, non-limiting Examples.

### EXAMPLE 1

Mushroom Strains and Growing Conditions

A commercial A. bisporus strain U3 (Sylvan, U.K.) and a carboxin resistant A. bisporus mutant C54-carb.8 were used in this work. Vegetative mycelium was produced on sterile compost at 25° C. and fully colonised compost (21 days) was frozen in liquid nitrogen. Mushrooms were grown in trays according to commercial practice at the Horticulture Research International mushroom cropping unit. Sporophores were produced in synchronous weekly flushes and mushroom fruit bodies from second flush were harvested at developmental stages one to seven and flash frozen. For tissue expression analysis, stage 4 mushrooms were dissected into stipe (upper and lower), cap (pileus trama), skin (pilei pellis) and gills (lamellae) and frozen. All frozen samples were stored at −80° C.

Bacterial Strains, Vectors and Phagemid/Cosmid DNA Extraction

Escherichia coli strains XL-1 Blue and XLOLR (Stratagene) were used for the preparation and propagation of cDNA clones. Phagemid and Cosmid DNA extractions were carried out using the Tip 20 plasmid DNA extraction kit (Qiagen).

Preparation of an Ordered Library of Random cDNA Clones

Total RNA extraction and poly (A)⁺ RNA isolation were carried out as described previously. Using 5 μg poly (A)⁺ RNA, cDNA libraries (ZAP EXPRESS, Stratagene) were constructed from veil break stage mushrooms, and mushrooms were harvested and stored for two days. Mass excision of the cDNA libraries was performed according to the manufacturer's (Stratagene) instructions. Clear single colonies (cDNA clones in pBK-CMV) were picked and ordered in microtitre plates containing 200 μI media 96 broth with Kanamycin (50 μg ml⁻¹) in each well.

Differential Screening

Duplicate nylon membranes containing DNA from the clones were prepared for differential screening. For preparing probes cDNA was generated from 5 μg poly (A)⁺ RNA using the Ready-to-Go T-primed cDNA synthesis kit (Amersham Pharmacia Biotech). Total cDNA's were labelled with [α-³²P] dCTP using the Redi-prime random labelling kit (Amersham Pharmacia Biotech). One set each of the membranes containing 3500 cDNA clones from the veil break stage mushrooms were hybridised with the cDNA probe from the veil break stage mushrooms and the cDNA probe from button stage mushrooms. Putative differentially expressed cDNA clones were re-screened to reduce false positives.

Northern Analysis

Total RNA was extracted from compost colonised vegetative mycelium and seven different developmental stages of the mushroom, freshly harvested button stage mushroom and samples stored from 1-5 days as well as from the stipe, cap and gill tissue. Equal quantities (15 μg) of total RNA for each sample were electrophoresed and Northern blots prepared as described elsewhere. cDNA randomly labelled with [α-³²P] dCTP, as described above, was used as the probe.

Genomic Library Screening and Identification of the Gene

For isolating cosmid clones containing the gene, a genomic library constructed from a carboxin resistant mutant of A. bisporus C54-carb.8 in cosmid vector Lawrist was used. Preliminary screening of DNA pools from 56 microtitre plates (96 clones each, ca. 5376) was done by PCR using primers designed from the cDNA sequence. Individual cosmid clones containing the gene were identified by probing colony blots of the 96 clones in each of the positive pools.

Nucleotide Sequence Determination and Analysis

cDNA's in the phagemid vector pBK-CMV were initially sequenced using the vector primers T3 and T7 and the full double strand sequence was obtained using additional primers synthesised from known sequence. Genomic sequence was generated from cosmid clones by primer walking, where the initial sequencing was by done using the cDNA primers and further primers for sequencing were designed from known sequence. Nucleotide sequences were determined using the ABI automated DNA sequencing technology. Sequencing reactions were carried by thermal cycling using the ABI Prism™ BigDye terminator cycle sequencing kit (ABI-Perkin Elmer) as per manufacturers' instructions. Editing and assembling of the sequence data were done using the programmes within the DNASTAR package (Lasergene software, Dnastar Inc.). Homology searches and nucleotide and amino acid sequence comparisons were made using a suite of software available on the WWW, particularly ExPASy (www.expasy.com) and other linked sites.

### EXAMPLE 2

Constructs

Constructs are prepared to maximise expression of specific proteins through the use of homologous regulatory sequences from the mushroom Agaricus bisporus. The sequences comprise (see FIG. 5):

promoter sequences P: 5′ untranslated regions preceding A. bisporus genes previously shown to be highly expressed in specific mushroom tissues or developmental stages;

leader sequences L (optional): specific N-terminal regions of proteins that encompass the ATG nucleotide start codon for the Methionine amino acid and a defined number of other amino acids implemented in the cellular ‘targeting’ of proteins;

intron sequences I (optional): the protein coding sequence is interrupted with a short region(s) of non-coding sequence. Intron sequences are naturally occurring in many eukaryotes and have been defined in all A. bisporus genes hitherto characterised. In the absence of L sequences the I cassette encompasses an ATG start codon that precedes the intron sequences;

gene/protein sequences G: coding sequences for the desired heterologous protein and encompassing a stop codon;

terminator region T: 3′ untranslated regions following defined genes from A. bisporus, or other fungi, and implemented in the accurate termination and stability of gene transcripts.

P, L I, G and T sequences are engineered from defined nucleic acids using the ‘polymerase chain reaction’ with defined oligonucleotide primers to introduce specific and novel restriction enzyme sites that facilitate the assembly of constructs through molecular cloning protocols. An example construct is shown in FIG. 5 with restriction enzyme sites represented by number 1-6. Therefore, cassette P with engineered restriction sites 1 at the 5′ end and 2 at the 3′ end is ligated with cassette L with engineered sites 2 (or compatible overhang sequence) at the 5′ end and 3 at the 3′ end.

P, (L), I, G and T cassettes are assembled in a plasmid vector (for example pBluescript2) and amplified at appropriate stages in E. coli using appropriate molecular cloning protocols.

Promoter (P) Sequences Used in Heterologous Protein Constructs

The ABST1 promoter sequence is shown in SEQ ID NO. 12. The RAFE promoter sequence is shown in SEQ ID NO. 13.

Strategy for Development of Promoter—Intron Cassette

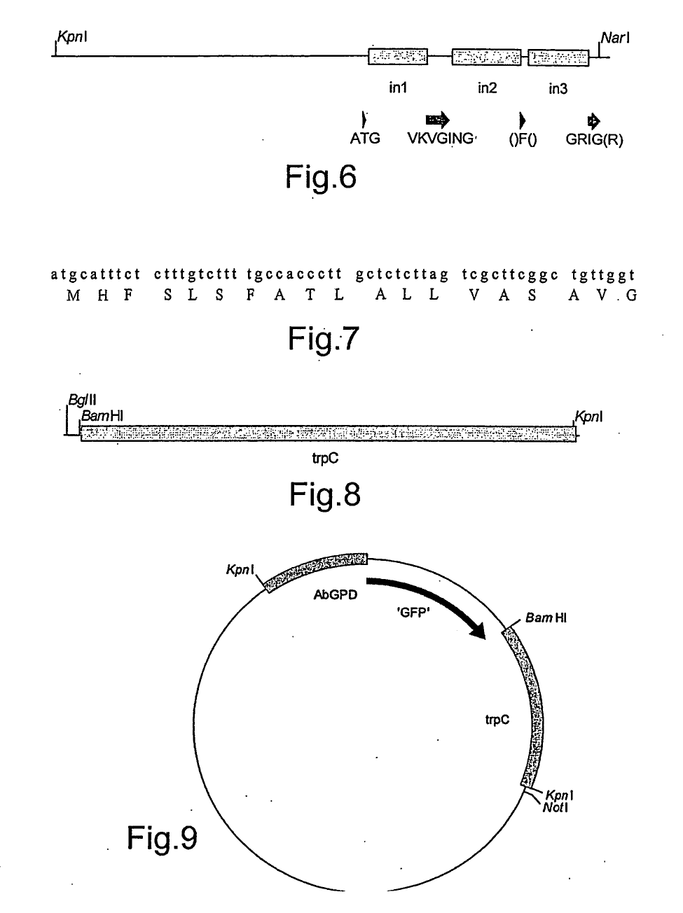

A. bisporus GPD promoter 5′UTR sequences ATG start codon and three naturally occurring introns (in1-3; lower case). An Engineered PCR product (486 nucleotides) with restriction sites KpnI and NarI and introns is shown in SEQ ID NO. 14 and in FIG. 6.

Leader (L) Sequence

An example of a suitable leader sequence is the SPR leader sequence, comprising 57 nucleotides (SEQ ID NO. 15) encoding 19 amino acids (SEQ ID NO. 16), and deposited as Genbank (NCBI)Accession Number Y13805. The sequence alignment of the nucleotide and amino acids is given in FIG. 7.

Intron (I) Sequences

In addition to GPD introns referred to above, numerous A. bisporus introns have been characterised that can be used as intron cassettes (see tables 2 to 5, below). These introns have been established by comparison of partial cDNA and genomic DNA sequences. The introns are described by their gene of origin (i.e. putative gene product) and Genbank (NCBI) cDNA accession number. For each gene, introns are numbered from the 5′ end. The first identified introns of each gene are theoretical and unconfirmed (cDNA sequence not available at 5′ end and therefore identified by comparison of gene sequence with sequences of similar genes from other organisms) and are not included in this list.

Terminator (T) Sequences

The abst1 terminator sequence is given in SEQ ID NO. 35. The rafe terminator sequence is given in SEQ ID NO. 36.

Strategy for Development of Terminator (T) Cassette

A. nidulans trpC terminator 3′UTR as an engineered PCR product (comprising 800 nucleotides) with introduced restriction sites BglII and KpnI, is given in SEQ ID NO. 37, and shown diagrammatically in FIG. 8.

Construct Strategy for Expression of GFP Protein

Construct in pBluescript plasmid combines A. bisporus GPD promoter with introns (as detailed above; P) fused with eGFP gene and A. nidulans trpC terminator (as described above; T), and represented in FIG. 9. Engineered KpnI sites are used to excise expression cassette from pBluescript and transfer to Agrobacterium binary vector harbouring hygromycin (hph) resistance selectable marker. T-DNA introduced into A. bisporus by Agro-transfection contains hph and heterologous protein (GFP) as a pair of divergently transcribed genes.

In the accompanying Sequence Listing, it will be noted as follows:

Organism: Agaricus bisporus

Gene designations (tentative): abst1 (sugar transporter); rafe (putative riboflavin aldehyde forming enzyme gene); mag2 (unidentified morphogenesis associated gene)

Gene identification: By differential screening of a cDNA library (3500 random clones) from stage 4 mushrooms

Genomic clones: From a genomic library of A. bisporus strain C54-carb8 in cosmid vector Lawrist (ca. 30-40 kb genomic fragments)

abst1: Up-regulated (more than 100 fold) during mushroom development, abundantly expressed through stages 4-7 (later stages of mushroom development), ca. 0.6% transcripts at stage 4, represented by 20 clones in the differential library, 1.7 kb transcript

rafe: Up-regulated (up to 50 fold) during mushroom development, abundantly expressed through stages 4-7, 0.7 kb transcript

mag2: Up-regulated (up to 30 fold) during mushroom development, ca. 0.6% transcripts at stage 4, represented by seven clones in the differential library, comparable levels of expression in stipe and cap tissue, ca. 0.7 kb transcript

The following Table provides an index of the accompanying SEQ ID NO's, and gives an indication as to their identity. The sequence provided in SEQ ID NO 4 was obtained on the basis of single strand analysis and is for guidance only, as there are some errors and unknown nucleotides. The promoter sequence of SEQ ID NO. 12 is preferred, as it is shorter, and does not comprise as many restriction sites. In addition, it was sequenced as a double strand.

TABLE 1

| Sequence description | SEQ ID NO |
|---|---|
| Abst1 cDNA sequence | 1 |
| Abst1 protein encoded by ORF of SEQ ID NO 1 | 2 |
| abst1 Genomic sequence | 3 |
| abst1 Promoter region (approx. 1000 bases upstream | 4 |
| of ORF). | |
| abst1 Terminator region (approx. 700 bases | 5 |
| downstream of ORF | |
| rafe cDNA sequence | 6 |
| Rafe protein encoded by ORF of SEQ ID NO2 | 7 |
| rafe Genomic Sequence | 8 |
| rafe Promoter region (approx. 1000 bases upstream of | 9 |
| ORF) | |
| rafe Terminator region (approx. 1200 bases | 10 |
| downstream of ORF): | |
| . mag2: Partial cDNA sequence | 11 |
| abst 1 promoter sequence | 12 |
| Rafe promoter sequence | 13 |
| Engineered PCR product (486 nt) with restriction sites | 14 |
| KpnI and NarI comprising A. bisporus GPD promoter, | |
| 5′UTR sequences, ATG start codon and three naturally | |
| occurring introns. | |
| SPR leader sequence (nucleotide sequence) | 15 |
| SPR leader sequence (amino acid sequence) | 16 |
| abst 1 terminator sequence | 35 |
| rafe terminator sequence | 36 |
| A. nidulans trpC terminator 3′UTR as engineered PCR | 37 |
| product (800 nt) with introduced restriction sites Bg/II | |
| and KpnI | |

TABLE 2
ASL-Argininosuccinate lyase (Genbank Accession no. AJ271691)

| Intron | SEQ ID NO. |
|---|---|
| ASL intron 3 | 17 |
| ASL intron 4 | 18 |
| ASL intron 5 | 19 |
| ASL intron 6 | 20 |

TABLE 3
CYPI-Cytochrome P450 I (Genbank Accession no. AJ271707)

| Intron | SEQ ID NO. |
|---|---|
| CYPII intron 6 | 21 |
| CYPII intron 7 | 22 |
| CYPII intron 8 | 23 |
| CYPII intron 9 | 24 |
| CYPII intron 10 | 25 |

TABLE 4
SOD-Superoxide Dismutase (Genbank Accession no. AJ271694)

| Intron | SEQ ID NO. |
|---|---|
| SOD intron 2 | 26 |
| SOD intron | 27 |

TABLE 5
LAP-Leucine Aminopeptidase (Genbank Accession no. AJ271690)

| Intron | SEQ ID NO. |
|---|---|
| LAP intron 3 | 28 |
| LAP intron 4 | 29 |
| LAP intron 5 | 30 |
| LAP intron 6 | 31 |
| LAP intron 7 | 32 |

TABLE 6
MET-Metallothionein (Genbank Accession no. AJ271695)

| Intron | SEQ ID NO. |
|---|---|
| MET intron 2 | 33 |
| MET intron 3 | 34 |

1. A filamentous fungus transformed with a heterologous sequence of DNA, the fungus being capable of expressing the heterologous DNA, characterised in that the heterologous DNA is under the control of a filamentous fungus transcription promoter which is substantially active later than stage 1 of the development of the fruiting body of the fungus and which is substantially up-regulated during development of the fruiting body, between the button stage, later than stage 1, and the veil-break stage.

2. A fungus according to claim 1, wherein the promoter is active only from veil-break onwards during the development of the fruiting body of the fungus.

3. A fungus according to claim 1, wherein the promoter is active only during stages 4 to 7 of the development of the fruiting body of the fungus.

4. A fungus according to claim 1, which is A. bisporus.

5. A fungus according to claim 1, wherein the promoter is obtainable from the cDNA sequence of SEQ. ID NO.11.

6. A fungus according to claim 1, wherein the promoter comprises the sequence of SEQ ID NO 12, or a mutation or variant thereof, or a sequence which hybridises thereto under conditions of at least 60% sequence identity.

7. A fungus according to claim 1, wherein the promoter comprises the sequence of SEQ ID NO 13, or a mutation or variant thereof, or a sequence which hybridises thereto under conditions of at least 60% sequence identity.

8. A fungus according to claim 1, wherein the DNA is operably linked with a terminator comprising the sequence of SEQ ID NO 35, or a mutation or variant thereof, or a sequence which hybridises thereto under conditions of at least 60% sequence identity.

9. A fungus according to claim 1, wherein the DNA is operably linked with a terminator comprising the sequence of SEQ ID NO 36, or a mutation or variant thereof, or a sequence which hybridises thereto under conditions of at least 60% sequence identity.

10. A fungus according to claim 1, wherein the DNA is operably linked with a promoter comprising the sequence of SEQ ID NO. 12 and a terminator comprising the sequence of SEQ ID NO 35, or a mutation or variant of either, or a sequence which hybridises thereto under conditions of at least 60% sequence identity.

11. A fungus according to claim 1, wherein the DNA is operably linked with a promoter comprising the sequence of SEQ ID NO. 13 and a terminator comprising the sequence of SEQ ID NO 36, or a mutation or variant of either, or a sequence which hybridises thereto under conditions of at least 60% sequence identity.

12. A fungus according to claim 1, wherein a selectable marker is linked with the heterologous DNA.

13. A fungus according to claim 1, wherein the heterologous DNA is native DNA.

14. A fungus according to claim 1, wherein the heterologous DNA is selected such as to affect characteristics of mushroom crop production.

15. A fungus according to claim 1, wherein the heterologous DNA encodes: antibodies, including other diagnostic material; secondary metabolites, such as lectins, pesticidal compounds such as Bacillus thuringiensis toxin (Bt toxin); therapeutic compounds such as vaccines, steroids, heterocyclic organic compounds; biological macromolecules, such as interferon, endostatin and insulin; and medical enzymes, such as thrombolytics and cerebrosidases.

16. A method for the production of a substance expressible by a DNA sequence, wherein the sequence is operably associated with a filamentous fungus transcription promoter as defined in claim 1, the sequence and promoter being expressibly incorporated in a filamentous fungus, the fungus being cultured to fruition and the product being harvested.

17. A method for the production of a substance expressible by a DNA sequence, wherein the sequence is operably associated with a filamentous fungus transcription promoter as defined in claim 1, the sequence and promoter being expressibly incorporated in a filamentous fungus, the fungus being cultured to fruition and the product being harvested, and wherein the DNA and/or fungus is as defined in claim 1.


SEQUENCE LISTING (37 sequences)

SEQ ID NO: 1 - DNA, 1736 bp, Agaricus bisporus
  CDS (19)..(1674)
ggctgagctctattcatcatggcgtcggaacgacagattgaagaacttcccagttataagtacgcctatattttgacggcatcggcttgcttgggaagtgtgtttcacgggtgggatgtaggccttataggaggcatactctcgctacggtcatttcaagaatatctcgggatcaatacaaaaaatgccgtcaagaaagcgattctagacggaaacatcatctctgtgctccaagccggatgtttttttggcgcgcttggaaccggatatctctctagtcgattcggccgaagaccctgtcttattgcatctggtattgtgtatataactggcggtttgctgcaatgcactgtcggtttgggaccctcgcaagctgctgctctacacgtgttctatattggcaggttcatttctggtatcggtgttgggatggtgtccactctcgtgcctttgtatatttcggagtgtgtccctaggactatacgcgggcgctgtactggaacactccaatttgcgactaacagtggtctgatgctgggcttctgggtcaactacagcgtgtcgaaaaacgtgccctttggtgaaatgcaatggcgaattccgttaattatccaaatgattccgagcctcttgttcatcatagccatgtttttccaaccagaatcgccgagatggcttgttgaacacgggaaacacaaggaagctgcgacggtactggcgcgtactggcggcaaggatgttgatcatcctagtgttgtacagacactggaggagatcaagcaagaatttgtggcgagtaaacaaccatcgtttttaaagcagattcgcctggtcggtgaatcgagggctgttgccctgaggtgctttataccaccgctagtgatgttcttccagcagtggacgggtacaaatgccatcaacctttatagtcccgaagtattccgtcatcttggaatccatggcaccagcggggctctcttcgctactggtgtttatggcgtggtgaaggttgtttcagttgcacttgccctcacttttgctgtcgaacgctttggacgcaagagagggttgatttttggtggtatcggccaagcacttatgatgttttggttgggaggttatagtgccacccaccaagacggtactgtcagtcctgcgagtcatgtttccattgttgcactctacttgtatggtgcattcttctccatgggatggggcccattaccatgggtcgtcgctggagaggttgcacctaaccatgtccgctccttcgccctctccatcgccgttggaactcattggctcttcgggtttgtgatatcaaaagtgacgccaattatgttggaccgtatcaaatatggcacattcctactcttcggattctgttgcatgatagtagcgacatgggcttatttctgtctacctgagacaagtgggttcgctctggaggacatcaaatatctgttcgagcgagacgtcatcattcgttcattgcaggacgctcccggtggaaaaatattcttgggggggaggcgtgtggaatctgtagcttcgttgaaagagaggcgcgttggagtcgctggtgagcagggtgagaagataactggtctaaattcggaattggaagatgtttcctcaaaaaaatcaacattgaaggaaacttcatccgtttgatatatagtctccaaattctattgtaatgccattttcccaattcaaaaaaaaaaaaaaaaaaa

SEQ ID NO: 2 - protein, 551 aa, Agaricus bisporus
MASERQIEELPSYKYAYILTASACLGSVFHGWDVGLIGGILSLRSFQEYLGINTKNAVKKAILDGNIISVLQAGCFFGALGTGYLSSRFGRRPCLIASGIVYITGGLLQCTVGLGPSQAAALHVFYIGRFISGIGVGMVSTLVPLYISECVPRTIRGRCTGTLQFATNSGLMLGFWVNYSVSKNVPFGEMQWRIPLIIQMIPSLLFIIAMFFQPESPRWLVEHGKHKEAATVLARTGGKDVDHPSVVQTLEEIKQEFVASKQPSFLKQIRLVGESRAVALRCFIPPLVMFFQQWTGTNAINLYSPEVFRHLGIHGTSGALFATGVYGVVKVVSVALALTFAVERFGRKRGLIFGGIGQALMMFWLGGYSATHQDGTVSPASHVSIVALYLYGAFFSMGWGPLPWVVAGEVAPNHVRSFALSIAVGTHWLFGFVISKVTPIMLDRIKYGTFLLFGFCCMIVATWAYFCLPETSGFALEDIKYLFERDVIIRSLQDAPGGKIFLGGRRVESVASLKERRVGVAGEQGEKITGLNSELEDVSSKKSTLKETSSV

SEQ ID NO: 3 - DNA, 3638 bp, Agaricus bisporus
  misc_feature (3)..(3): n is an unknown nucleotide
gtncgatgggttcntctgggttaagttgcacgacgctttcctttttcttttatggcctgtctgcccttttaacgctttatctttcggcagccatggatgtccttcgtcaccgtattatcactcttaatcgtggtggtgagcacatggaaaggttcattatcgtcatcccatgacgcggtgcaaaattcgtcattcagagtggaaccgatactaggagaggattttgaaaaggctatcgtatcttccgatccattcactccaacgacatctatatcgggttctcaagctacgataaccgttcctcacgcatcacattcttctctgattgtaatgaccgaagaccgcggttccacccctaatattcgaagagcttgttgccaatgtcaaaaaaggcttttgggcaaaccgggatggcttatcgagcctccagccccagcagctaacatcgggcagagaaggaaaattcatcgggcggttgaattatcaccgtttggttcctgagtcatctgnragatgtacgcagatggtgataccgtgtttgattggcgccgttggagaagaactatattattcgatggattttttgttcgagtttgacacagagacagagatgatagaggtttgctattgatgtagcaaaggatcatttgacgatggcgcatagggcgatggttatctttatgtctggaattataatatgtattgttccccacttttcttttatatttattaatactaattggaagtttcagttgttggatgagcaaagttggtgcagatagaaactagaattcggattcccatatctgaggtaccttttccttccgctggcaatcctggccacttcgacgtggtgacgcagagggcgcgtgctattgttagcacatgccatawggatcgacgttgcctctcgtacttcgcgcctaggctcgctcatgcctcgatgcatcttttcaattcgggcgttgcgtctcccaggtgcctgttaaaagggcgaactttagtgtaattgtactaacacagtccctcgggctgagctctattcatcatggcgtcggaacgacagattgaagaacttcccagttataagtacgcctatattttgacggcatcggcttgcttgggaagtgtgtttcacgggtgggatgtgcaagtatcctttwgacgctggctcatcccttgtttgctgatggttaatgtgtagaggccttataggaggcatactctcgctacggtcatttcaagaatatctcgggatcaatacaaaaaatgccgtcaagaaagcgattctagacggaaacatcatctctgtgctccaagccggatgttttgtaagtcgtagcactcgttcgaccagcttactttcttttactaacaacgtgttgacctagttttggcgcgcttggaaccggatatctctctagtcgattcggccgaagaccctgtcttattgcatctggtattgtgtatataactggcggtttgctgcaatgcactgtcggtttgggaccctcgcaagctgctgctctacacgtgttctatattggcaggttcatttctggtatcggtgttgggatggtgtccactctcgtgcctttgtatatttcggagtgtgtccctaggactatacgcgggcgctgtactggaacactccaatttgcgactaacagtggtctgatgctgggctgtaagtgtaccttttgttatgctccggggacgatactaaaagtagtctgtagtctgggtcaactacagcgtgtcgaaaaacgtgccctttggtgaaatgcaatggcgaattccgttaattatccagtacgttattggatgcaaagtgaagcatatgctgaaattagacttgtgtcgtactttccagaatgattccgagcctcttgttcatcatagccatgtttttccaaccagaatcgccgagatggcttgttgaacacgggaaacacaaggaagctgcgacggtactggcgcgtactggcggcaaggatgttgatcatcctagtgttgtacagacactggaggagatcaagcaagaatttgtggcgagtaaacaaccatcgtttttaaagcagattcgcctggtcggtgaatcgagggctgttgccytgaggwgctttataccaccgctagtgatgttcttccagcagtggacgggtacaaatgccatcaacctttatagtcccgaagtattccgtcatcttggaatccatggcaycagckrkgmwstcttcsctamtsgtgtttatkgmgtggtrmaggttgtttcagttgcacttgccctcacttttgctgtcgaacgctttggacgcaagagagggttgatttttggtggtatcggccaagcacttatgatgttttggttgggaggttatagtgccacccaccaagacggtactgtcagtcctgcgagtcatgtttccattgttgcactctacttgtatggtgcattcttctccactgaagaatggttcctgttabcatgggtcgtcgctggagaggttgcacctaaccatgtccgctccttcgccctctccatcgccgttggaactcattggctcttcgggtttgtgatatcaaaagtgacgccaattatgttggaccgtatcaaatatggcacattcctactcttcggattctgttgcatgatagtagcgacatgggcttatttctgtctacctgagacaagtgggttcgctctggaggacatcaaatatctgttcgagcgagacgtcatcattcgttcattgcaggacgctcccggtggaaaaatattcttgggggggaggcgtgtggaatctgtagcttcgttgaaagagaggcgcgttggagtcgctggtgagcagggtgagaagataactggtctaaattcggaattggaagatgtttcctcaaaaaaatcaacattgaaggaaacttcatccgtttgatatatagtctccaaattctattgtaatgccattttcccaattcaaaaggacccgctctcgaaccgggtcagatgcaattttggtcagcaatggtttatgttgtttccccgtaagtatgcactagaagagaacaaaacgtcactatttgctcaatgcaggatgcacctggcgagataatattcttgcggtgaagtcgaacaacgtctgtagtcctgtaaaaatatacagtgagtagagggatgatgccgatgtggaaggaagcaaccgattacgattcggatttggccatgagacggccgctcttgaacagaccaacaatatccctttaaatttaatacagaattactcaatatgcttccaagtatttcgattcctcgaattccgtctggccgcggcatgagcatggacaggcggacagaagaggctatcgttgtattgcttcatcaagcgaccctgactagtgacttcaggcatgatcatgcgcttagcaatctgtcccttcaagtcgagtccccgaattcaacagctttcaacaagtcgtgattatttgacccccgactggaatcaaattggctcttcaaatttcaaaccttcaatgcttcatgcttcatgcgtcatgacgcaagctgtcaattttcattttccagttcggtcccattctcactctcgcccctcstaatgtcttccagaaaatggcttccc

SEQ ID NO: 4 - DNA, 1033 bp, Agaricus bisporus
  misc_feature (495)..(495): n is an unknown nucleotide
ctgggttaagttgcacgacgctttcctttttcttttatggcctgtctgcccttttaacgctttatctttcggcagccatggatgtccttcgtcaccgtattatcactcttaatcgtggtggtgagcacatggaaaggttcattatcgtcatcccatgacgcggtgcaaaattcgtcattcagagtggaaccgatactaggagaggattttgaaaaggctatcgtatcttccgatccattcactccaacgacatctatatcgggttctcaagctacgataaccgttcctcacgcatcacattcttctctgattgtaatgaccgaagaccgcggttccacccctaatattcgaagagcttgttgccaatgtcaaaaaaggcttttgggcaaaccgggatggcttatcgagcctccagccccagcagctaacatcgggcagagaaggaaaattcatcgggcggttgaattatcaccgtttggttcctgagtcatctgnragatgtacgcagatggtgataccgtgtttgattggcgccgttggagaagaactatattattcgatggattttttgttcgagtttgacacagagacagagatgatagaggtttgctattgatgtagcaaaggatcatttgacgatggcgcatagggcgatggttatctttatgtctggaattataatatgtattgttccccacttttcttttatatttattaatactaattggaagtttcagttgttggatgagcaaagttggtgcagatagaaactagaattcggattcccatatctgaggtaccttttccttccgctggcaatcctggccacttcgacgtggtgacgcagagggcgcgtgctattgttagcacatgccatawggatcgacgttgcctctcgtacttcgcgcctaggctcgctcatgcctcgatgcatcttttcaattcgggcgttgcgtctcccaggtgcctgttaaaagggcgaactttagtgtaattgtactaacacagtccctcgggctgagctctattcatc

SEQ ID NO: 5 - DNA, 702 bp, Agaricus bisporus
tatatagtctccaaattctattgtaatgccattttcccaattcaaaaggacccgctctcgaaccgggtcagatgcaattttggtcagcaatggtttatgttgtttccccgtaagtatgcactagaagagaacaaaacgtcactatttgctcaatgcaggatgcacctggcgagataatattcttgcggtgaagtcgaacaacgtctgtagtcctgtaaaaatatacagtgagtagagggatgatgccgatgtggaaggaagcaaccgattacgattcggatttggccatgagacggccgctcttgaacagaccaacaatatccctttaaatttaatacagaattactcaatatgcttccaagtatttcgattcctcgaattccgtctggccgcggcatgagcatggacaggcggacagaagaggctatcgttgtattgcttcatcaagcgaccctgactagtgacttcaggcatgatcatgcgcttagcaatctgtcccttcaagtcgagtccccgaattcaacagctttcaacaagtcgtgattatttgacccccgactggaatcaaattggctcttcaaatttcaaaccttcaatgcttcatgcttcatgcgtcatgacgcaagctgtcaattttcattttccagttcggtcccattctcactctcgcccctcstaatgtcttccagaaaatggcttccc

SEQ ID NO: 6 - DNA, 689 bp, Agaricus bisporus
  CDS (147)..(521)
cgcctacttttataccaaccccaaatccaaaggttgaaaaaaaaatttcgacaaggatttatatatccatccatccgcgacactttcccgtttgattctatcccttagtctttccttctccccctttccttcttcacttcaccttgctctaaccgaaagtaaacctttccgcgatgaaattctccaattctctatccgctctcctcgtatccgcaaatctcatgttggcagcgaaggcctacaaaggagatgccaccttttatgatcctggtctgggagcttgtggccatacgaatcaggctcatgaacttgtcgttgcccttccatcagccaaatacggcagcggagaccattgttccaagcatgtcggcatccactacaaaggcaaatacgtgaaagccaaagtagtcgacaaatgtcccggttgtggttcgaacgatttagacatctcaccaaccgcattctctcacttagccagtcaagacctcggccgtatcaaagtagattgggaatttctctgatatcccattttcaatcccttacacgaaatctgtatttgtagaagaaagtcatgacgttatatagatcacttacatagatcttcaggttttcgtagatcgacgaccgacgctcttaaatatttatttcccgtttttcygtttttgttttaaaaaaaaaaaaaaaaaaaa

SEQ ID NO: 7 - protein, 122 aa, Agaricus bisporus
PKVNLSAMKFSNSLSALLVSANLMLAAKAYKGDATFYDPGLGACGHTNQAHELVVALPSAKYGSGDHCSKHVGIHYKGKYVKAKVVDKCPGCGSNDLDISPTAFSHLASQDLGRIKVDWEFL

SEQ ID NO: 8 - DNA, 2782 bp, Agaricus bisporus
  misc_feature (2774)..(2774): n is an unknown nucleotide
ctgcgaattggcataagcacttraactttcgtcttcctcactctcttcaggagattgagactgcatcggtgcaagcgagggttgccgtaccgccctttgagaccccgaaacaagggattcgtcaagagcatcaagaagactagaacgaccggctgtttttccacccgacatcatagcacaaactgtcataaacccgtgttcaaaggggaaaaacaggcagagagaaggaagggacgcgtcgcgatgaaatgctcattaacctgaatgacaaacttccgcwaataacaatttaattaaaaataaaaatcacgaggtgacaaacaggggtgtttacctccattcgactgcatcctggctcttcccttccatagaactgtcgtccatgcatgcaccgctagcatcgcactgctctgactcgcataaccttaaaacgcgtggaccccctgttcggacggccggttcaggatccggggctcaggacacagtaaaatcacaaaaactcatactttgagagatatgacttctcgacttgcgccttcgatggacggacaaattatccccaggtaccggatctgtgacaccgaattagtgcgcgatattatatatgacttttgacgggcgtctcatacgaccgctcaagtccttggggatggagaatgtcacctcctggtccaccgggcccagagcattacccggtcattaatctagcgcttcttgcatgcactcctgcatgatcaccccacgcggccgcgttttatcggacatataaggaacaagattccataggtagtggatcccctactccacctcccgcctacttttataccaaccccaaatccaaaggttgaaaaaaaaatttcgacaaggatttatatatccatccatccgcgacactttctcgtttgattctatcccttagtctttccttctccccctttccttcttcacttcaccttgctctaaccgaaagtaaacctttccgcgatgaaattctccaattctctatccgctctcctcgtatccgcgaatctcatgttggcagcgaaggcctacaaaggagatggtcagttatgacagtgtcgcccatcctatcgtcaatgatgctgattttgttctcccacctttttagccaccttttatgatcctggtgtacgtttttcttacacttttcaccaagattcctcacccactggtttcaaaattagctgggagcttgtggccatacgaatcaggctcatgaacttgttgttgcccttccatcagccaaatacggcaacggagaccattgttccaagcatgtcggcatccactgtacgtttcctccttctccccacttctcaaaaatcaaaattttactcattgtaacaagacaaaggcaaatacgtgaaagtcaaagtagtcgacaaatgtcccggctgtggttcgaacgatctagacatctcaccaaccgcattctctcagttagccagccaagacctcggccgtatcaaagtagattgggaatttatctaatatcccatttcaatcccttacacgaaatctgtacttgtagtcttagaagaaagtcatgacgttatatagatcacttacatagatcttcaggttttcgtagatcgacgaccgacgctcttaaatatttatttacagtttttctgttttttgttttattgtcgcttggatataaggtggtatactttgatatgattgcctacacacatatatcaacacagttttagttatatcaacatcaaaacatcagtcaaggaaaacaaagagcgaacgataaacatcagcacaagtatgtcagattatggtccaagaacgcgaaaagaagttcgcaaaagaacagaacactatcgaaaagtgcagatacataggtcacacaattaacgacttcccggaatagttccctccaacctcttatcgcgactactagcaccaacggtaacaccaaaagtaccttcaggcctcctccatccctgtgcattcacatcccaaatactcaaatcatacctcgacaaggtcatttttacattcctagtctctccagggccaatcggtacagagtcgaaaccgcgtagcacggaaggaggttctccagcagattcagggaagttaatgtagagttggggagactcggcacccaaaagtcgaccggtattcttgacgttgaaggaaacctcgtacaaaggacgatggagcctggaaaccggtcagcgtgaaattgttgrttagagaaccctcggcgcttaccaagaagcgatagaaccaccttcaacttgcgggcttgcatgtccttgatcccattccctagccgcatcaacttctctacctttgacgtgcactttcttaatagatatcttcgagtaatcaaacttcgtgtaactcaagccaaaaccaaattcaaaccgaggctcaatgccattctgcaagagaaaattcagtaagcgaacgtttccgagagaatcaagcagcactggcttacagcgtcgaagtgccggtaatcgatttgcaaacgatcatcgtatggtatagaaagaatatctcgaataccatctccggggataacttgggccgaataatcttcaatccgtttggcaatggtatatggaagccttccggatgggttccaatctccatataggacatcggtcaacgaatttcccgcttctgtcccggtgccccagcccaaagaacctggaaatagtcaacaccggcgttcatcacacaagtagaagatacgaacagcagtgacattaggatgattgatccanggttcaag

SEQ ID NO: 9 - DNA, 984 bp, Agaricus bisporus
ctgcgaattggcataagcacttraactttcgtcttcctcactctcttcaggagattgagactgcatcggtgcaagcgagggttgccgtaccgccctttgagaccccgaaacaagggattcgtcaagagcatcaagaagactagaacgaccggctgtttttccacccgacatcatagcacaaactgtcataaacccgtgttcaaaggggaaaaacaggcagagagaaggaagggacgcgtcgcgatgaaatgctcattaacctgaatgacaaacttccgcwaataacaatttaattaaaaataaaaatcacgaggtgacaaacaggggtgtttacctccattcgactgcatcctggctcttcccttccatagaactgtcgtccatgcatgcaccgctagcatcgcactgctctgactcgcataaccttaaaacgcgtggaccccctgttcggacggccggttcaggatccggggctcaggacacagtaaaatcacaaaaactcatactttgagagatatgacttctcgacttgcgccttcgatggacggacaaattatccccaggtaccggatctgtgacaccgaattagtgcgcgatattatatatgacttttgacgggcgtctcatacgaccgctcaagtccttggggatggagaatgtcacctcctggtccaccgggcccagagcattacccggtcattaatctagcgcttcttgcatgcactcctgcatgatcaccccacgcggccgcgttttatcggacatataaggaacaagattccataggtagtggatcccctactccacctcccgcctacttttataccaaccccaaatccaaaggttgaaaaaaaaatttcgacaaggatttatatatccatccatccgcgacactttctcgtttgattctatcccttagtctttccttctccccctttccttcttcacttcaccttgctctaaccgaaagtaaacctttccgcg

SEQ ID NO: 10 - DNA, 1270 bp, Agaricus bisporus
  misc_feature (1262)..(1262): n is an unknown nucleotide
tatcccatttcaatcccttacacgaaatctgtacttgtagtcttagaagaaagtcatgacgttatatagatcacttacatagatcttcaggttttcgtagatcgacgaccgacgctcttaaatatttatttacagtttttctgttttttgttttattgtcgcttggatataaggtggtatactttgatatgattgcctacacacatatatcaacacagttttagttatatcaacatcaaaacatcagtcaaggaaaacaaagagcgaacgataaacatcagcacaagtatgtcagattatggtccaagaacgcgaaaagaagttcgcaaaagaacagaacactatcgaaaagtgcagatacataggtcacacaattaacgacttcccggaatagttccctccaacctcttatcgcgactactagcaccaacggtaacaccaaaagtaccttcaggcctcctccatccctgtgcattcacatcccaaatactcaaatcatacctcgacaaggtcatttttacattcctagtctctccagggccaatcggtacagagtcgaaaccgcgtagcacggaaggaggttctccagcagattcagggaagttaatgtagagttggggagactcggcacccaaaagtcgaccggtattcttgacgttgaaggaaacctcgtacaaaggacgatggagcctggaaaccggtcagcgtgaaattgttgrttagagaaccctcggcgcttaccaagaagcgatagaaccaccttcaacttgcgggcttgcatgtccttgatcccattccctagccgcatcaacttctctacctttgacgtgcactttcttaatagatatcttcgagtaatcaaacttcgtgtaactcaagccaaaaccaaattcaaaccgaggctcaatgccattctgcaagagaaaattcagtaagcgaacgtttccgagagaatcaagcagcactggcttacagcgtcgaagtgccggtaatcgatttgcaaacgatcatcgtatggtatagaaagaatatctcgaataccatctccggggataacttgggccgaataatcttcaatccgtttggcaatggtatatggaagccttccggatgggttccaatctccatataggacatcggtcaacgaatttcccgcttctgtcccggtgccccagcccaaagaacctggaaatagtcaacaccggcgttcatcacacaagtagaagatacgaacagcagtgacattaggatgattgatccanggttcaag

SEQ ID NO: 11 - DNA, 835 bp, Agaricus bisporus
ccttgccgttttccagaagctgcgaccagtcctcggaggagggggaactaaagatgtcgaaagcagcagggtgtgtgacatagcgggaagaaagggtgatgatgatgagaacgaggggtacgatgaggacggcccatctgaattgacggccaacgcgacgtttccggtcgtgagaggacatggcaaaggagacggggggaggggcgagggtggcggaggaggtgctcgtgccgaattcggcacgagctcaccatgaaattcgcaactgctctcctcgcctgccttactgctgctgctagcgctcarcgcgtcctcatcggatcccctcctgaccaagcaaatctttctgctggccagaacactacgattcaaattgtactcccgaatttccaatcgtcttcgcaagaagttgcggtagtgcttgggatcacgtcctgcgccgctgctccctgccctgctccagccgatacgatgggtcgtatcctttacagcggtcatttcaacccgcagagagatcctgcaatgcccgcaatgcaagcctacgaaaatttcacggtcttcttgccggagaacctgcctaagggcgcggcgcagattaacgtttaccatgtcgcacttatcggggccggtctcatgccatggaacgagacgttgtccaccacagctttgattcagtaattccatcaggatttgaaatggacctttagtagtttactgttttgctatcgaacgattcgrataattacctgagatcaggtcggtgactgaggcccgtcggagtgctaccataatggcataataaaattatactcagctgaaaaaaaaaaaaaaaaaa

SEQ ID NO: 12 - DNA, 770 bp, Agaricus bisporus
tcgggttctcaagctacgataaccgttcctcacgcatcacattcttctctgattgtaatgaccgaagaccgcggttccacccctaatattcgaagagcttgttgccaatgtcaaaaaaggcttttgggcaaaccgggatggcttatcgagcctccagccccagcagctaacatcgggcagagaaggaaaatcatcggcgttgaattatcaccgtttggttcctgagtcatctggagatgtacgcagatggtgataccgtgtttgattggcgccgttggagaagaactatattattcgatggattttttgttcgagtttgacacagagacagagatgatagaggtttgctattgatgtagcaaaggatcatttgacgatggcgcatagggcgatggttatctttatgtctggaattataatatgtattgttccccacttttcttttatatttattaatactaattggaagtttcagttgttggatgagcaaagttggtgcagatagaaactagaattcggattcccatatctgaggtaccttttccttccgctggcaatcctggccacttcgacgtggtgacgcagagggcgcgtgctattgttagcacatgccatatggatcgacgttgcctctcgtacttcgcgcctaggctcgctcatgcctcgatgcatctttcaattcgggcgttgcgtctcccaggtgcctgttaaaagggcgaactttagtgtaattgtactaacacagtccctcgggctgagctctattcatc

SEQ ID NO: 13 - DNA, 703 bp, Agaricus bisporus
ataacaatttaattaaaaataaaaatcacgaggtgacaaacaggggtgtttacctccattcgactgcatcctggctcttcccttccatagaactgtcgtccatgcatgcaccgctagcatcgcactgctctgactcgcataaccttaaaacgcgtggaccccctgttcggacggccggttcaggatccggggctcaggacacagtaaaatcacaaaaactcatactttgagagatatgacttctcgacttgcgccttcgatggacggacaaattatccccaggtaccggatctgtgacaccgaattagtgcgcgatattatatatgacttttgacgggcgtctcatacgaccgctcaagtccttggggatggagaatgtcacctcctggtccaccgggcccagagcattacccggtcattaatctagcgcttcttgcatgcactcctgcatgatcaccccacgcggccgcgttttatcggacatataaggaacaagattccataggtagtggatcccctactccacctcccgcctacttttataccaaccccaaatccaaaggttgaaaaaaaaatttcgacaaggatttatatatccatccatccgcgacactttctcgtttgattctatcccttagtctttccttctccccctttccttcttcacttcaccttgctctaaccgaaagtaaacctttccgcg

SEQ ID NO: 14 - DNA, 486 bp, Agaricus bisporus
  misc_feature (4)..(9): Restriction site for KpnI
cggggtaccgaggtccgcaagtagattgaaagttcagtacgtttttaacaatagagcattctcgaggcttgcgtcattctgtgtcaggctagcagtttataagcgttgaggatctagagctgctgtttccgcgtctcgaatgttctcggtgtttaggggttagcaatctgatatgataataatttgtgatgacatcgatagtacaaaaaccccaattccggtcacatccaccatctccgttttctcccatctacacacaacaagcttatcgccatggtttgtctctcgcttgcataccatccagcagctcactgatgtcgacttgtaggttaaagttggaatcaacgggtaagtgtttttgtcgtcgcgctgtggttccggatcatctcagactttgggtgtcttgcagtttcggtgagtgaccaccctgcattctggctatatgcgtgatactgaccatcgctcaaggtcgtatcggcgccggcc

SEQ ID NO: 15 - DNA, 57 bp, Agaricus bisporus
  CDS (1)..(57)
atgcatttctctttgtcttttgccacccttgctctcttagtcgcttcggctgttggt

SEQ ID NO: 16 - protein, 19 aa, Agaricus bisporus
MHFSLSFATLALLVASAVG

SEQ ID NO: 17 - DNA, 45 bp, Agaricus bisporus
gtgggattatggttagccaaatggtcgtagctaattatcttgcag

SEQ ID NO: 18 - DNA, 47 bp, Agaricus bisporus
gtacgtaggcaagtggttcccaaaaagcaccagctaacacaacgtag

SEQ ID NO: 19 - DNA, 48 bp, Agaricus bisporus
gtacgttgaatcgtacaagaaagtgtaatcatcctgactttctatcag

SEQ ID NO: 20 - DNA, 62 bp, Agaricus bisporus
gtaagcaccggctgcgttcgcacacccgtcttgtgaaaagtcgtctcatgaatatcgcccag

SEQ ID NO: 21 - DNA, 51 bp, Agaricus bisporus
gtaattaacctcatcattattgatcctttccatgcttacagctgttatcag

SEQ ID NO: 22 - DNA, 52 bp, Agaricus bisporus
gtgcgttcttctcttgtcctatcacaaattctgacgccgcaggacttgccag

SEQ ID NO: 23 - DNA, 63 bp, Agaricus bisporus
gcaagcttctcttgtcacaatgttaacggcgaggggtctgactcccttggttgtttttgttag

SEQ ID NO: 24 - DNA, 48 bp, Agaricus bisporus
gtacgctatagcttgcaaggatggacatatctaatcggggacgtgtag

SEQ ID NO: 25 - DNA, 61 bp, Agaricus bisporus
gtgggttccatcatctcttgagttatgccgcggctgactgatcatgtttctaatacttcag

SEQ ID NO: 26 - DNA, 46 bp, Agaricus bisporus
gtgcgtcatgtccgtatcatctactctcatactaatgcgcatatag

SEQ ID NO: 27 - DNA, 53 bp, Agaricus bisporus
gtaggtcccttttatttttggtccgattgcgtcattcatgtctatatatgcag

SEQ ID NO: 28 - DNA, 48 bp, Agaricus bisporus
gtaggttttattcatccctctgttcttatcagcttgacatccttcaag

SEQ ID NO: 29 - DNA, 53 bp, Agaricus bisporus
gtaagcataaggagttgctgcaggcgacccatagcgttctaattagctgctag

SEQ ID NO: 30 - DNA, 52 bp, Agaricus bisporus
gtatgttggaactcaccgatgcgctctttgttgattttatttttctacatag

SEQ ID NO: 31 - DNA, 50 bp, Agaricus bisporus
gtgggtgatcattcgaggttgtcttcctgtgtattgataaggtttgctag

SEQ ID NO: 32 - DNA, 45 bp, Agaricus bisporus
gtcagtactagtttgttttctcttacaccttctcattctttgcag

SEQ ID NO: 33 - DNA, 56 bp, Agaricus bisporus
gtgagcactgcaatatggtatagcttggaaagcctttatttatacagaacatccag

SEQ ID NO: 34 - DNA, 55 bp, Agaricus bisporus
gtgagtgaagatagttctcatgtgagatccttgtactaattgccgaacgtcgtag

SEQ ID NO: 35 - DNA, 690 bp, Agaricus bisporus
tatatagtctccaaattctattgtaatgccattttcccaattcaaaaggacccgctctcgaaccgggtcagatgcaattttggtcagcaatggtttatgttgtttccccgtaagtatgcactagaagagaacaaaacgtcactatttgctcaatgcaggatgcacctggcgagataatattcttgcggtgaagtcgaacaacgtctgtagtcctgtaaaaatatacagtgagtagagggatgatgccgatgtggaaggaagcaaccgattacgattcggatttggccatgagacggccgctcttgaacagaccaacaatatccctttaaatttaatacagaattactcaatatgcttccaagtatttcgattcctcgaattccgtctggccgcggcatgagcatggacaggcggacagaagaggctatcgttgtattgcttcatcagcgaccctgactagtgacttcaggcatgatcatgcgcttagcaatctgtcccttcaagtcgagtccccgaattcaacagcttcaacaagtcgtgattatttgacccccgactggaatcaaattggctcttcaaatttcaaacttcaatgcttcatgcttcatgcgtcatgacgcaagctgtcaattttcattttccagttcggtcccattctcactctcgcccctcctaatgtcttccagaaaa

SEQ ID NO: 36 - DNA, 910 bp, Agaricus bisporus
tatcccatttcaatcccttacacgaaatctgtacttgtagtcttagaagaaagtcatgacgttatatagatcacttacatagatcttcaggttttcgtagatcgacgaccgacgctcttaaatatttatttacagtttttctgttttttgttttattgtcgcttggatataaggtggtatactttgatatgattgcctacacacatatatcaacacagttttagttatatcaacatcaaaacatcagtcaaggaaaacaaagagcgaacgataaacatcagcacaagtatgtcagattatggtccaagaacgcgaaaagaagttcgcaaaagaacagaacactatcgaaaagtgcagatacataggtcacacaattaacgacttcccggaatagttccctccaacctcttatcgcgactactagcaccaacggtaacaccaaaagtaccttcaggcctcctccatccctgtgcattcacatcccaaatactcaaatcatacctcgacaaggtcatttttacattcctagtctctccagggccaatcggtacagagtcgaaaccgcgtagcacggaaggaggttctccagcagattcagggaagttaatgtagagttggggagactcggcacccaaaagtcgaccggtattcttgacgttgaaggaaacctcgtacaaaggacgatggagcctggaaaccggtcagcgtgaaattgttgattagagaaccctcggcgcttaccaagaagcgatagaaccaccttcaacttgcgggcttgcatgtccttgatcccattccctagccgcatcaacttctctacctttgacgtgcactttcttaatagatatcttcgagtaatcaaacttcgtgtaactcaagccaaaaccaaattcaaaccgaggctcaatgc

SEQ ID NO: 37 - DNA, 800 bp, Agaricus bisporus
  misc_feature (5)..(9): Restriction site for BglII
gaatagatctgatgccgaccgcgggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagaagtcctcgtgtactgtgtaagcgcccactccacatctccacggtacctgcag